(19) United States
(12) Patent Application Publication
(10) Pub. No.: US 2020/0141897 A1
(43) Pub. Date: May 7, 2020

(54) FET-BASED SENSING OF CO2 FOR ENVIRONMENTAL AND FOOD APPLICATIONS

(71) Applicant: International Business Machines Corporation, Armonk, NY (US)

(72) Inventors: Bharat Kumar, Tarrytown, NY (US); Damon B. Farmer, White Plains, NY (US)

(21) Appl. No.: 16/178,092

(22) Filed: Nov. 1, 2018

(51) Int. Cl. G01N 27/414; H01L 51/05; H01L 51/00; G01N 33/00

(52) U.S. Cl. CPC G01N 27/4141; G01N 33/004; H01L 51/0003; H01L 51/0508

(57) ABSTRACT

A FET-based CO₂ sensor is provided in which the conducting channel of the FET device is coated with a chemical compound that has a high degree of CO₂ selectivity and reversibility.

### BACKGROUND

The present application relates to a carbon dioxide (CO₂) sensor which can be used in a variety of environmental and food applications. More particularly, the present application relates to a field effect transistor (FET)-based sensor that is selective for CO₂ sensing.

A CO₂ sensor is a device that is used for the measurement of carbon dioxide, CO₂, gas in a wide variety of environmental applications such as, for example, green house gas emissions, and food safety inspections. The most common principles for CO₂ sensors are infrared gas sensors.

Infrared gas sensors are spectroscopic sensors that are employed to detect CO₂ in a gaseous environment. The key components of an infrared gas sensor are an infrared light source, a light tube, an interference (i.e., wavelength) filter, and an infrared detector. In such infrared gas sensors, the gas is pumped, or diffused, into the light tube, and the electronics measure the absorption of the characteristic wavelength of light.

New developments include using micro-electromechanical systems to bring down the costs of infrared gas sensors and to create smaller devices. Infrared CO₂ sensors are also used for dissolved CO₂ for applications such as beverage carbonation, pharmaceutical fermentation and CO₂ sequestration applications. In this case, the infrared sensors are typically mated to an ATR (attenuated total reflection) optic device, and the CO₂ gas is measured in situ.

FET-based sensors are known and have the unique advantage of lower cost and longer durability than infrared sensors. Despite these advantages, there are currently no sensors based on FETs for selective CO₂ sensing. As such, there is a need for providing a FET-based sensor that has a high selectivity for sensing CO₂.

### SUMMARY

A FET-based CO₂ sensor is provided in which the conducting channel of the FET device is coated with a chemical compound that has a high degree of CO₂ selectivity and reversibility. The FET-based CO₂ sensor of the present application can be used in a wide variety of applications including environmental applications and food applications.

In one aspect of the present application, a FET-based CO₂ sensor is provided. In one embodiment, the FET-based CO₂ sensor includes a dielectric material located on a surface of a conductive material layer. A first electrode is located on a first portion of the dielectric material and, a second electrode, which is spaced apart from the first electrode, is located on a second portion of the dielectric material. A conducting channel is located between the first and second electrodes. In accordance with the present application, the conducting channel is coated with a chemical compound that is selective for CO₂ sensing.

In another aspect of the present application, a method of forming a FET-based CO₂ sensor is provided. In one embodiment, the method includes forming a structure that includes a dielectric material located on a surface of a conductive material layer, a first electrode located on a first portion of the dielectric material, a second electrode spaced apart from the first electrode and located on a second portion of the dielectric material, and a conducting channel located between the first and second electrodes. Next, the conducting channel is coated with a chemical compound that is selective for CO₂ sensing.

In either aspect of the present application, the chemical compound that is used for coating the conducting channel has a high degree of CO₂ selectivity and reversibility. In one embodiment, the chemical compound is an organic compound that contains an amine functionality such as, for example, monoethanolamine (MEA), diethanolamine (DEA), 2-amino-2-methyl-1-propanol (AMP), 2-(aminoethyl)ethanolamine (AEEA), or piperazine. The organic compound may be a monomer or in polymeric form.

### BRIEF DESCRIPTION OF THE DRAWINGS

FIG. 1 is a cross sectional view of an exemplary structure including a dielectric material located on a surface of a conductive material layer, a first electrode located on a first portion of the dielectric material, a second electrode spaced apart from the first electrode and located on a second portion of the dielectric material, and a conducting channel located between the first and second electrodes that can be employed in the present application.

FIG. 2 is a cross sectional view of the exemplary structure of FIG. 1 after coating the conducting channel with a chemical compound that is selective for CO₂ sensing.

FIG. 3 is a graph of normalized current (μAmpere (A)) vs. time of a FET-based CO₂ sensor of the present application showing that the sensor can be used for detecting CO₂ and the reversibility of the sensor.

FIG. 4 is a graph of normalized current (μAmpere (A)) vs. time of a FET-based CO₂ sensor of the present application showing that the sensor has CO₂ selectivity and the reversibility of the sensor.

### DETAILED DESCRIPTION

The present application will now be described in greater detail by referring to the following discussion and drawings that accompany the present application. It is noted that the drawings of the present application are provided for illustrative purposes only and, as such, the drawings are not drawn to scale. It is also noted that like and corresponding elements are referred to by like reference numerals.

In the following description, numerous specific details are set forth, such as particular structures, components, materials, dimensions, processing steps and techniques, in order to provide an understanding of the various embodiments of the present application. However, it will be appreciated by one of ordinary skill in the art that the various embodiments of the present application may be practiced without these specific details. In other instances, well-known structures or processing steps have not been described in detail in order to avoid obscuring the present application.

It will be understood that when an element as a layer, region or substrate is referred to as being “on” or “over” another element, it can be directly on the other element or intervening elements may also be present. In contrast, when an element is referred to as being “directly on” or “directly over” another element, there are no intervening elements present. It will also be understood that when an element is referred to as being “beneath” or “under” another element, it can be directly beneath or under the other element, or intervening elements may be present. In contrast, when an element is referred to as being “directly beneath” or “directly under” another element, there are no intervening elements present.

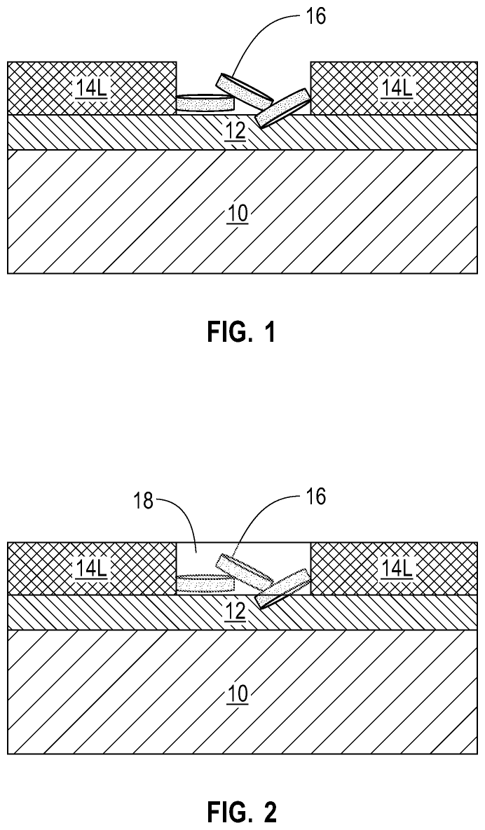

Referring first to FIG. 1, there is illustrated an exemplary structure including a dielectric material 12 located on a surface of a conductive material layer 10, a first electrode 14L located on a first portion of the dielectric material 12, a second electrode 14R spaced apart from the first electrode 14L and located on a second portion of the dielectric material 12, and a conducting channel 16 located between the first and second electrodes (14L, 14R) that can be employed in the present application. As is shown, the first electrode 14L is located on a first side of the conducting channel 16, and the second electrode 14R is located on a second side of the conducting channel 16 that is opposite the first side.

It is noted that although the present application describes and illustrates a structure including a single conducting channel 16 located between first and second electrodes (14L, 14R), the present application contemplates embodiments wherein a plurality of conducting channels are present and each conducting channel of the plurality of conducting channel is in contact with a pair of spaced apart electrodes. Thus, an array of FET-based sensors can be provided.

It is further noted that the drawings of the present application illustrate an area in which a FET-based CO₂ sensor in accordance with the present application will be provided. Non-FET-based CO₂ sensor device areas, which may include conventional semiconductor devices (i.e., transistors, capacitors, and/or resistors) may be located laterally adjacent the area that contains the FET-based CO₂ sensor of the present application. Thus, and in some embodiment of the present application the present application can provide a semiconductor chip that contains a FET-based CO₂ sensor that is integrated on a same semiconductor substrate with conventional semiconductor devices.

The conductive material layer 10 that can be employed in the present application includes any material that can serve as, i.e., function as, a gate electrode. In one embodiment, the conductive material layer 10 that can be used in the present application includes one or more semiconductor materials having semiconducting properties. Examples of semiconductor materials that may be used as the conductive material layer 10 include, but are not limited to, silicon (Si), a silicon germanium (SiGe) alloy, a silicon germanium carbide (SiGeC) alloy, germanium (Ge), a III/V compound semiconductor, or a II/VI compound semiconductor. A III-V compound semiconductor is a semiconductor material that contains at least one element from Group III of the Periodic Table of Elements and at least one element from element from Group V of the Periodic Table of Elements. A II-VI compound semiconductor is a semiconductor material that contains at least one element from Group II of the Periodic Table of Elements and at least one element from element from Group VI of the Periodic Table of Elements.

The semiconductor material that provides the conductive material layer 10 is typically doped with a p-type or n-type dopant. The term “n-type” denotes the addition of impurities that contributes free electrons to an intrinsic semiconductor. In a silicon containing semiconductor material, examples of n-type dopants, i.e., impurities, include, but are not limited to, antimony, arsenic and phosphorous. The term “p-type” denotes the addition of impurities to an intrinsic semiconductor that creates deficiencies of valence electrons. In a silicon-containing semiconductor material, examples of p-type dopants, i.e., impurities, include, but are not limited to, boron, aluminum, gallium and indium.

In another embodiment, the conductive material layer 10 is a metal (e.g., tungsten (W), titanium (Ti), tantalum (Ta), ruthenium (Ru), hafnium (Hf), zirconium (Zr), cobalt (Co), nickel (Ni), copper (Cu), aluminum (Al), platinum (Pt), tin (Sn), silver (Ag), gold (Au), a conducting metallic compound material (e.g., tantalum nitride (TaN), titanium nitride (TiN), tantalum carbide (TaC), titanium carbide (TiC), titanium aluminum carbide (TiAlC), tungsten silicide (WSi), tungsten nitride (WN), ruthenium oxide (RuO₂), cobalt silicide (CoSi), nickel silicide (NiSi)), transition metal aluminides (e.g., Ti₃Al, ZrAl), TaC, TaMgC, conductive carbon, graphene, or any suitable combination of these materials.

The dielectric material 12, which is formed upon a surface of the conductive material layer 10, can be composed of a dielectric oxide, nitride, and/or oxynitride. In one embodiment, the dielectric material 12 can be composed of silicon dioxide. In another embodiment, the dielectric material 12 can be composed of a high-k material having a dielectric constant greater than 4.0. Exemplary high-k dielectrics include, but are not limited to, HfO₂, ZrO₂, La₂O₃, Al₂O₃, TiO₂, SrTiO₃, LaAlO₃, Y₂O₃, HfOₓN_{y}, ZrOₓN_{y}, La₂OₓN_{y}, Al₂OₓN_{y}, TiOₓN_{y}, SrTiOₓN_{y}, LaAlOₓN_{y}, Y₂OₓN_{y}, SiON, SiNₓ, a silicate thereof, and an alloy thereof. Each value of x is independently from 0.5 to 3 and each value of y is independently from 0 to 2. In some embodiments, a multilayered dielectric structure composed of different dielectric materials, e.g., silicon dioxide, and a high-k gate dielectric, can be formed and used as the dielectric material 12.

The dielectric material 12 can be formed by any deposition process including, for example, chemical vapor deposition (CVD), plasma enhanced chemical vapor deposition (PECVD), physical vapor deposition (PVD), sputtering, or atomic layer deposition (ALD). In one embodiment of the present application, the dielectric material 12 can have a thickness in a range from 1 nm to 10 nm. Other thicknesses that are lesser than, or greater than, the aforementioned thickness range can also be employed for the dielectric material 12.

The first and second electrodes (14L, 14R) are composed of any conductive material including, for example, a doped semiconductor material (e.g., doped silicon, doped germanium or a doped silicon germanium alloy), an elemental metal (e.g., tungsten, titanium, tantalum, aluminum, nickel, ruthenium, palladium and platinum), an alloy of at least two elemental metals, an elemental metal nitride (e.g., tungsten nitride, aluminum nitride, and titanium nitride), an elemental metal silicide (e.g., tungsten silicide, nickel silicide, and titanium silicide) or multilayered combinations thereof. In one embodiment, the first electrode 14L is composed of a same conductive material as the second electrode 14R. In one example, the first and second electrodes (14L, 14R) are composed of palladium. In another embodiment, the first electrode 14L is composed of a conductive material that is compositionally different from the second electrode 14R. In one example, the first electrode 14L is composed of palladium and the second electrode 14R is composed of nickel.

The conductive material used in providing the first and second electrodes (14L, 14R) can be formed utilizing a deposition process including, for example, chemical vapor deposition (CVD), plasma enhanced chemical vapor deposition (PECVD), physical vapor deposition (PVD), sputtering, atomic layer deposition (ALD) or other like deposition processes. When a metal silicide is formed, a conventional silicidation process is employed. In one embodiment, each of the first and second electrodes (14L, 14R) can have a thickness, i.e., height, from 50 nm to 200 nm. Other thicknesses that are lesser than, or greater than, the aforementioned thickness range can also be employed for the first and second electrodes (14L, 14R). Typically, but not necessarily always, the first and second electrodes (14L, 14R) have a same height.

The conducting channel 16 that is employed in the present application is located between the first and second electrodes (14L, 14R) and has one surface that contacts the first electrode 14L and another surface that contacts the second electrode 14R. In some embodiments, an entirety of the conducting channel 16 may be located directly on the dielectric material 12. In other embodiments, at least one portion of the conducting channel 16 is located directly on the dielectric material 12. In yet further embodiments, the entirety of the conducting channel 16 is suspended above the dielectric material 12.

In one embodiment, the conducting channel 16 is composed of a semiconductor material, as defined above. In some embodiments, the semiconductor material 16 is doped with an n-type or p-type dopant, as also defined above. The concentration of dopant within the semiconductor material that provides the conducting channel 16 can be from 1E14 atoms/cm³ to 1E18 atoms/cm³.

In such an embodiment, the semiconductor material that provides the conducting channel 16 may be formed utilizing an epitaxial growth process utilizing any well-known precursor gas or gas mixture. Carrier gases like hydrogen, nitrogen, helium and argon can be used. In some embodiments, a dopant can added to the precursor gas or gas mixture to provide a doped semiconductor material that can be employed as the conducting channel 16. In other embodiments, a non-doped semiconductor material layer can be epitaxially grown and thereafter the dopant can be added to the non-doped semiconductor material utilizing ion implantation or gas phase doping.

In another embodiment, the semiconductor material that provides the conducting channel 16 may present on a handle wafer and then a bonding process is used to bond the semiconductor material to the dielectric material 12. After bonding, the handle substrate is typically removed. In such an embodiment, the first and second electrodes (14L, 14R) are formed after the bonding process and the removal of the handle substrate.

In another embodiment, the conducting channel 16 is composed of one or more carbon nanotubes. Carbon nanotubes (CNTs) are allotropes of carbon with a cylindrical nanostructure. The CNTs that can be used as the conducting channel 16 may be single-walled or multi-walled. In one embodiment, a single CNT is employed as the conducting channel 16. In another embodiment, a network of CNTs can be employed as the conducting channel 16. In such an embodiment, the CNTs within the network of CNTs are typically randomly orientated, as is shown, for example, in FIG. 1 of the present application.

The CNT(s) that can be employed as the conducting channel 16 can be formed utilizing any well-known process including, for example, arc discharge, laser ablation, or chemical vapor deposition (CVD). Other techniques for forming CNTs can also be used in the present application.

The exemplary structure shown in FIG. 1 can be formed utilizing any well-known technique. In one embodiment, the exemplary structure shown in FIG. 1 can be formed by first depositing the dielectric material 12 on a surface of the conductive material layer 10. Next, a conducting channel 16 is formed by deposition and photolithography, followed, by formation of the first and second electrodes (14L, 14R). The formation of the first and second electrodes (14L, 14R) typically includes deposition of a conductive material and photolithography. In another embodiment, the first and second electrodes (14L, 14R) are formed prior to the conducting channel 16, and thereafter the conducting channel 16 is formed.

Referring now to FIG. 2, there is illustrated the exemplary structure of FIG. 1 after coating the conducting channel 16 with a chemical compound that is selective for CO₂ sensing. In FIG. 2, element 18 denotes a coating of the chemical compound that is selective for CO₂ sensing (hereinafter referred to as a chemical compound coating 18).

The chemical compound coating 18 that is employed in the present application can be composed of an organic compound that contains an amine functional group; the amine functional group exhibits a high affinity for bonding with CO₂. In one embodiment, the organic compound that contains the amine functional group is monoethanolamine (MEA), diethanolamine (DEA), 2-amino-2-methyl-1-propanol (AMP), 2-(aminoethyl)ethanolamine (AEEA), or piperazine. In some embodiments, the amine functional group is composed of a polymer containing monoethanolamine (MEA), a polymer containing diethanolamine (DEA), a polymer containing 2-amino-2-methyl-1-propanol (AMP), a polymer containing 2-(aminoethyl)ethanolamine (AEEA), or a polymer containing piperazine. In such an embodiment, the organic compounds containing the amine functionality can be incorporated into a polymer backbone using chemical methods well-known to those skilled in the art.

Other types of chemical compounds can also be used as the chemical compound coating 18 as long as the other types of chemical compounds exhibit a high degree of CO₂ sensing selectivity and reversibility. By “high degree of CO₂ sensing selectivity” it is meant that the sensor can detect CO₂ in the presence of other gases without the other gases interfering with the CO₂ detection. By “reversibility” it is meant after CO₂ has been removed from the sensor, the sensor is capable of returning back to its original setting.

The coating of the conducting channel 16 with the chemical compound that is selective for CO₂ sensing may be performed utilizing any well-known coating process such as, for example, spin-coating, drop casting, dip coating, or brush coating, which introduces the chemical compound to the conducting channel 16. Typically, the coating of the conducting channel 16 with the chemical compound that is selective for CO₂ sensing includes providing a solution that includes the chemical compound that is selective for CO₂ sensing. The solution may include a solvent such as, for example, an alcohol. Exemplary alcohols that may be used include ethanol, methanol, glycerol, ethylene glycol, propanol or any mixture of these alcohols. After the coating process has been performed, any residual solvent can then be removed utilizing a drying process.

The chemical compound coating 18 that is provided contacts the conducting channel 16 and may have a thickness from 1 nm to 20 nm. The chemical compound coating 18 may also contact physically exposed sidewall surfaces of the first and second electrodes (14L, 14R) and/or any physically exposed surface of the dielectric material 12. The chemical compound coating 18 may, or may not, fill in the entirety of the gap that is located between the first and second electrodes (14L, 14R).

The exemplary structure shown in FIG. 2 is a FET-based sensor that has a high degree of CO₂ selectivity as compared to a FET-based sensor without the chemical compound coating 18. Moreover, the FET-based sensor shown in FIG. 2 has a high degree of reversibility as compared to a FET-based sensor without the chemical compound coating 18.

The sensor of the present application works by supplying a constant voltage between the first and second electrodes (14L, 14R). This establishes a measurable electrical channel current between the first and second electrodes (14L, 14R). When CO₂ is introduced to the sensor, the CO₂ interacts with the CO₂-sensitive functional groups in chemical compound coating 18. The charge-transfer-doping supplied by this interaction causes the current in the conducting channel 16 to change. Monitoring changes in this current therefore allows CO₂ detection. The conductive material layer 10 can be used to determine the transconductance characteristics of the sensor sweeping the voltage on conductive material layer 10, while keeping the voltage between the first and second electrodes (14L, 14R) constant. Modulation of the conducting channel 16 current by doing this allows for the determination of the switching characteristics and dynamic range of the sensor, i.e., how much current modulation is attained with the introduction of a given gate voltage or given amount of doping from CO₂. This allows for evaluation of sensor sensitivity. Furthermore, voltage control of conductive material layer 10 allows for low power operation. The voltage on conductive material layer 10 can be set so that the conducting channel 16 is in the “off”, low current, state, thus drawing little power. High power is only needed when CO₂ sensing occurs, where the conducting channel 16 switches to the “on”, high current, state.

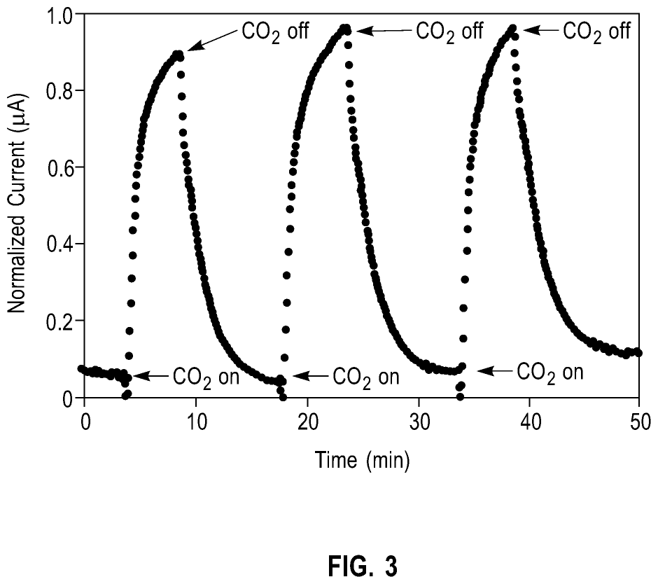

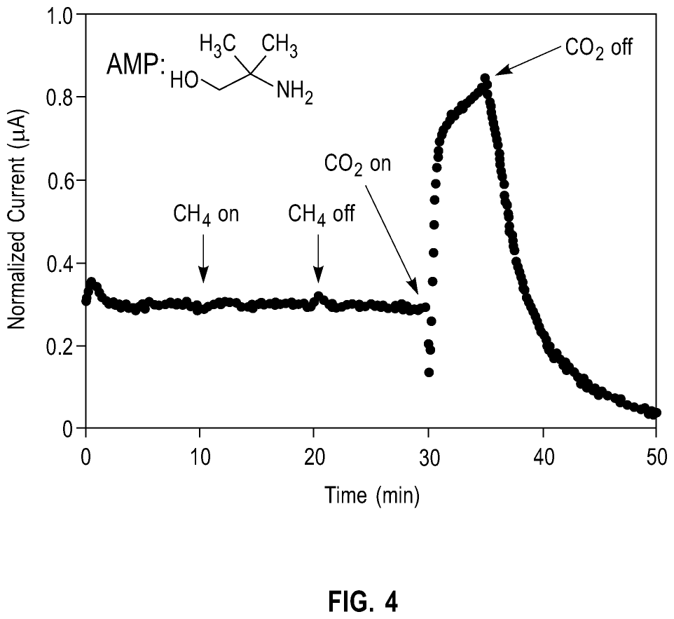

Referring now to FIGS. 3-4, there are shown graphs of normalized current (μAmpere (A)) vs. time of a FET-based CO₂ sensor of the present application showing that the sensor can be used for detecting CO₂ and the reversibility of the sensor. The FET-based CO₂ sensor that was employed in providing the data shown in FIGS. 3 and 4 was in accordance with the present application, and included a conducting channel composed of a network of carbon nanotubes that is located between palladium electrodes. The conducting channel of each FET-based CO₂ sensor was coated with 2-amino-2-methyl-1-propanol (AMP). The conductive material layer was silicon.

Notably, FIG. 3 shows that the FET-based CO₂ sensor of the present application had a high CO₂ sensitivity when CO₂ is present, while being reversible in the absence of CO₂. FIG. 4 shows that the FET-based CO₂ sensor of the present application had a high CO₂ sensitivity for CO₂ as compared with methane, CH₄. The measurements were taken in an enclosed container, through which different gases could be introduced and purged. In these experiments, the CO₂ concentration was 10% for CO₂ introduction and the CH₄ concentration was 100% for CH₄ introduction. The flow rate for both gases was 10 sccm and Ar was used as the purging gas. A random network, CNT film was used as the channel material and AMP was used as the functionalizing chemical. An AMP/ethanol solution was applied to the channel via drop casting. A voltage of −0.5V was applied between the first and second electrodes (14L, 14R) and a gate voltage of 0V was applied during the measurements.

The FET-based CO₂ sensor of the present application can be used to detect CO₂ in various applications including, but not limited to, environmental applications or food applications. In one application, the FET-based CO₂ sensor of the present application could be implemented in a ‘smart’ building to determine the level of CO₂ within a room that is present in the building.

While the present application has been particularly shown and described with respect to preferred embodiments thereof, it will be understood by those skilled in the art that the foregoing and other changes in forms and details may be made without departing from the spirit and scope of the present application. It is therefore intended that the present application not be limited to the exact forms and details described and illustrated, but fall within the scope of the appended claims.

What is claimed is:

1. A field effect transistor (FET)-based carbon dioxide (CO₂) sensor comprising:
a dielectric material located on a surface of a conductive material layer;
a first electrode located on a first portion of the dielectric material;
a second electrode spaced apart from the first electrode and located on a second portion of the dielectric material; and
a conducting channel located between the first and second electrodes, wherein the conducting channel is coated with a chemical compound that is selective for CO₂ sensing.

2. The FET-based CO₂ sensor of claim 1, wherein the chemical compound is an organic compound that contains an amine functional group.

3. The FET-based CO₂ sensor of claim 2, wherein the organic compound that contains the amine functional group comprises monoethanolamine (MEA), diethanolamine (DEA), 2-amino-2-methyl-1-propanol (AMP), 2-(aminoethyl)ethanolamine (AEEA), or piperazine.

4. The FET-based CO₂ sensor of claim 2, wherein the organic compound that contains the amine functional group comprises a polymer containing monoethanolamine (MEA), a polymer containing diethanolamine (DEA), a polymer containing 2-amino-2-methyl-1-propanol (AMP), a polymer containing 2-(aminoethyl)ethanolamine (AEEA), or a polymer containing piperazine.

5. The FET-based CO₂ sensor of claim 1, wherein the conducting channel is composed of a semiconductor material.

6. The FET-based CO₂ sensor of claim 1, wherein the conducting channel is composed of one or more carbon nanotubes.

7. The FET-based CO₂ sensor of claim 6, wherein the one or more carbon nanotubes comprise a network of carbon nanotubes.

8. A field effect transistor (FET)-based carbon dioxide (CO₂) sensor comprising:
a dielectric material located on a surface of a conductive material layer;
a first electrode located on a first portion of the dielectric material;
a second electrode spaced apart from the first electrode and located on a second portion of the dielectric material; and
a conducting channel composed of randomly oriented carbon nanotubes located between the first and second electrodes, wherein the conducting channel is coated with an organic compound that contains an amine functional group.

9. The FET-based CO₂ sensor of claim 8, wherein the organic compound that contains the amine functional group comprises monoethanolamine (MEA), diethanolamine (DEA), 2-amino-2-methyl-1-propanol (AMP), 2-(aminoethyl)ethanolamine (AEEA), or piperazine.

10. The FET-based CO₂ sensor of claim 8 wherein the organic compound that contains the amine functional group comprises a polymer containing monoethanolamine (MEA), a polymer containing diethanolamine (DEA), a polymer containing 2-amino-2-methyl-1-propanol (AMP), a polymer containing 2-(aminoethyl)ethanolamine (AEEA), or a polymer containing piperazine.

11. A method of forming a field effect transistor (FET)-based carbon dioxide (CO₂) sensor, the method comprising:
providing a structure that includes a dielectric material located on a surface of a conductive material layer, a first electrode located on a first portion of the dielectric material, a second electrode spaced apart from the first electrode and located on a second portion of the dielectric material, and a conducting channel located between the first and second electrodes; and
coating the conducting channel with a chemical compound that is selective for CO₂ sensing.

12. The method of claim 11, wherein the chemical compound is an organic compound that contains an amine functional group.

13. The method of claim 12, wherein the organic compound that contains the amine functional group comprises monoethanolamine (MEA), diethanolamine (DEA), 2-amino-2-methyl-1-propanol (AMP), 2-(aminoethyl)ethanolamine (AEEA), or piperazine.

14. The method of claim 12, wherein the organic compound that contains the amine functional group comprises a polymer containing monoethanolamine (MEA), a polymer containing diethanolamine (DEA), a polymer containing 2-amino-2-methyl-1-propanol (AMP), a polymer containing 2-(aminoethyl)ethanolamine (AEEA), or a polymer containing piperazine.

15. The method of claim 11, wherein the conducting channel is composed of a semiconductor material.

16. The method of claim 11, wherein the conducting channel is composed of one or more carbon nanotubes.

17. The method of claim 16, wherein the one or more carbon nanotubes comprise a network of carbon nanotubes.

18. The method of claim 17, wherein the network of carbon nanotubes is randomly orientated.

19. The method of claim 11, wherein the coating comprises:
providing a solution of the organic compound;
introducing the solution to the conducting channel to coat the conducting channel with the organic compound.

20. The method of claim 19, further comprising removing the solution.
